(19) United States
(12) Patent Application Publication
(10) Pub. No.: US 2008/0275079 A1
(43) Pub. Date: Nov. 6, 2008

(54) M3 Muscarinic Acetylcholine Receptor Antagonists

(75) Inventors: Jakob Busch-Petersen, King of Prussia, PA (US); Dramane Ibrahim Laine, King of Prussia, PA (US); Michael R. Palovich, King of Prussia, PA (US); Roderick S. Davis, King of Prussia, PA (US); Wei Fu, King of Prussia, PA (US); Haibo Xie, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED

(21) Appl. No.: 11/997,466

(22) PCT Filed: Aug. 2, 2006
(86) PCT No.: PCT/US2006/030153

§ 371 Date: Jan. 31, 2008

Related U.S. Application Data
(60) Provisional application No. 60/704,579, filed on Aug. 2, 2005.

(51) Int. Cl. A61K 31/437; C07D 451/02; A61P 17/00

(52) U.S. Cl. 514/304; 546/125

(57) ABSTRACT

Muscarinic Acetylcholine receptor antagonists and methods of using them are provided.

### FIELD OF THE INVENTION

This invention relates to novel thiazole aniline compounds, pharmaceutical compositions, processes for their preparation, and use thereof in treating M₃ muscarinic acetylcholine receptor mediated diseases.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs, and these receptors can mediate both inhibitory and excitatory actions. For example, in smooth muscle found in the airways, bladder and gastrointestinal tract, M₃ mAChRs mediate contractile responses. For review, please see (1).

Muscarinic acetylcholine receptor dysfunction has been noted in a variety of different pathophysiological states. For instance, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation. This mAChR dysfunction results in airway hyperreactivity mediated by increased stimulation of M₃ mAChRs. Similarly, inflammation of the gastrointestinal tract in inflammatory bowel disease (IBD) results in M₃ mAChR-mediated hypermotility (3). Incontinence due to bladder hypercontractility has also been demonstrated to be mediated through increased stimulation of M₃ mAChRs. Thus the identification of subtype-selective mAChR antagonists may be useful as therapeutics in these mAChR-mediated diseases.

Despite the large body of evidence supporting the use of anti-muscarinic receptor therapy for treatment of a variety of disease states, relatively few anti-muscarinic compounds are in use in the clinic. Thus, there remains a need for novel compounds that are capable of causing blockade at M₃ mAChRs. Conditions associated with an increase in stimulation of M₃ mAChRs, such as asthma, COPD, IBD and urinary incontinence would benefit by compounds that are inhibitors of mAChR binding.

### DESCRIPTION OF THE INVENTION

This invention provides for a method of treating a muscarinic acetylcholine receptor (mAChR) mediated disease, wherein acetylcholine binds to an M₃ mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I) and a pharmaceutical carrier or diluent.

Compounds of Formula (I) useful in the present invention are represented by the structures:

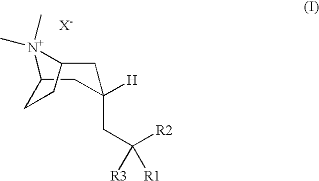

### Wherein:

R1 and R2 are independently selected from the following groups:

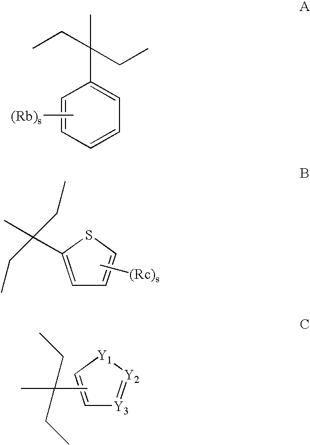

or 3-thienyl, pyridyl, benzyl, pyrimidyl, thiazolyl, isothiazolyl or C₃₋₇cycloalkyl, all of which may be optionally substituted;
R3 is hydrogen or hydroxy;
R4 and R5 are independently selected from the group consisting of hydrogen and optionally substituted C₁₋₄alkyl;
Rb is independently selected from the group consisting of halogen, hydroxy, cyano, nitro, dihalomethyl, trihalomethyl and NR4R5;
Rc is independently selected from the group consisting of C₁₋₄alkyl, halogen, hydroxy, cyano, nitro, dihalomethyl, trihalomethyl and NR4R5;
X— is a physiologically acceptable anion, such as chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate;

### Y1 is O or NR4;

Y2 and Y3 are independently selected from the group consisting of N and CH; and
s is an integer having a value of 1 to 3.
Illustrative compounds of Formula (I) include:
(3-Endo)-3-[2,2-Bis-(3-hydroxy-phenyl)-ethyl]-8,8-dimethyl-8-azonia-bicyclo[3.2.1]octane bromide;
(3-Endo)-3-[2,2-Bis-(3-chloro-phenyl)-ethyl]-8,8-dimethyl-8-azonia-bicyclo[3.2.1]octane iodide;
(3-Endo)-3-[2,2-Bis-(5-chloro-2-thienyl)-2-hydroxy-ethyl]-8,8-dimethyl-8-azonia-bicyclo[3.2.1]octane bromide;
(3-Endo)-1,1-Bis-(3-fluoro-phenyl)-2-(8,8-dimethyl-8-azonia-bicyclo[3.2.1]oct-3-yl)-ethanol iodide;
(3-Endo)-3-[2,2-Bis-(3-fluoro-phenyl)-ethyl]-8,8-dimethyl-8-azonia-bicyclo[3.2.1]octane iodide;
(3-Endo)-3-[2-(3-Chloro-phenyl)-2-phenyl-ethyl]-8,8-dimethyl-8-azonia-bicyclo[3.2.1]octane iodide;
(3-Endo)-1,1-bis(5-fluoro-2-methylphenyl)-2-(8,8-dimethyl-8-azoniabicyclo[3.2.1]oct-3-yl)ethanol bromide;
(3-Endo)-3-(2,2-Bis-(3-thienyl)ethyl)-8,8-dimethyl-8-azoniabicyclo[3.2.1]octane iodide;
(3-Endo)-3-[2-Hydroxy-2,2-bis-(3-methyl-2-thienyl)-ethyl]-8,8-dimethyl-8-azonia-bicyclo[3.2.1]octane bromide;
(3-Endo)-3-[2-Hydroxy-2,2-bis-(3-methoxy-phenyl)-ethyl]-8,8-dimethyl-8-azonia-bicyclo[3.2.1]octane iodide;
(3-Endo)-3-[2-Hydroxy-2,2-bis-(4-methyl-3-thienyl)-ethyl]-8,8-dimethyl-8-azonia-bicyclo[3.2.1]octane bromide;
(3-Endo)-3-[2-Hydroxy-2,2-bis-(5-methyl-2-thienyl)-ethyl]-8,8-dimethyl-8-azonia-bicyclo[3.2.1]octane bromide;
(3-Endo)-3-{2,2-Bis-[5-(1,1-difluoro-methyl)-2-thienyl]-2-hydroxy-ethyl}-8,8-dimethyl-8-azonia-bicyclo[3.2.1]octane bromide;
(3-Endo)-1,1-Bis-(3-thienyl)-2-(8,8-dimethyl-8-azoniabicyclo[3.2.1]oct-3-yl)ethanol iodide;
(3-Endo)-1,1-bis(3,4-difluorophenyl)-2-(8,8-dimethyl-8-azoniabicyclo[3.2.1]oct-3-yl)ethanol bromide,
(3-Endo)-3-[2,2-bis(3,4-difluorophenyl)ethyl]-8,8-dimethyl-8-azoniabicyclo[3.2.1]octane bromide;
(3-Endo)-1,1-bis(3,5-difluorophenyl)-2-(8,8-dimethyl-8-azoniabicyclo[3.2.1]oct-3-yl)ethanol bromide;
(3-Endo)-3-[2,2-bis(3,5-difluorophenyl)ethyl]-8,8-dimethyl-8-azoniabicyclo[3.2.1]octane bromide;
(3-Endo)-1,1-bis[5-fluoro-2-(methyloxy)phenyl]-2-(8,8-dimethyl-8-azoniabicyclo[3.2.1]oct-3-yl)ethanol bromide;
(3-Endo)-1,1-bis(3-fluoro-2-methylphenyl)-2-(8,8-dimethyl-8-azoniabicyclo[3.2.1]oct-3-yl)ethanol bromide;
(3-Endo)-3-[2,2-bis(5-fluoro-2-methylphenyl)ethyl]-8,8-dimethyl-8-azoniabicyclo[3.2.1]octane iodide;
(3-Endo)-1,1-dicyclohexyl-2-(8,8-dimethyl-8-azoniabicyclo[3.2.1]oct-3-yl)ethanol bromide;
(3-Endo)-1,1-dicyclopentyl-2-(8,8-dimethyl-8-azoniabicyclo[3.2.1]oct-3-yl)ethanol bromide.
(3-Endo)-1,3-bis(2-fluorophenyl)-2-[(8,8-dimethyl-8-azoniabicyclo[3.2.1]oct-3-yl)methyl]-2-propanol bromide;
2-[(3-Endo)-8,8-dimethyl-8-azoniabicyclo[3.2.1]oct-3-yl]-1,1-di-2-pyridinylethanol iodide;
(3-Endo)-1,1-Bis-(4-fluoro-phenyl)-2-(8,8-dimethyl-8-azonia-bicyclo[3.2.1]oct-3-yl)-ethanol iodide;
(3-Endo)-1,1-Bis-(4-chloro-phenyl)-2-(8,8-dimethyl-8-azonia-bicyclo[3.2.1]oct-3-yl)-ethanol iodide;
(3-Endo)-3-[2,2-Bis-(4-fluoro-phenyl)-ethyl]-8,8-dimethyl-8-azonia-bicyclo[3.2.1]octane iodide;
(3-Endo)-1,1-Bis-(3-chloro-phenyl)-2-(8,8-dimethyl-8-azonia-bicyclo[3.2.1]oct-3-yl)-ethanol iodide;
(3-Endo)-1-(2,3-Fluoro-phenyl)-2-(8,8-dimethyl-8-azonia-bicyclo[3.2.1]oct-3-yl)-1-phenyl-ethanol iodide; and
(3-Endo)-1-(2,3-Chloro-phenyl)-2-(8,8-dimethyl-8-azonia-bicyclo[3.2.1]oct-3-yl)-1-phenyl-ethanol iodide.

### Methods of Preparation

The compounds of Formula (I) may be obtained by utilizing synthetic procedures, some of which are illustrated in the Schemes below. The synthesis provided for these Schemes is applicable for producing compounds of formula (I) with a variety of different R1, R2 and R3.

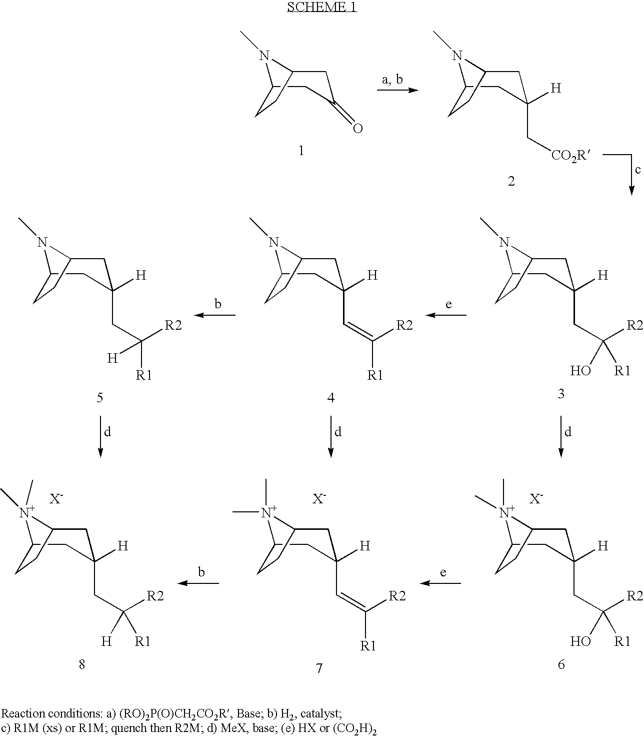

Azabicyclo ketones such as 1 can be prepared by a reaction known to those skilled in the art as a Robinson-Schopf condensation (For a general procedure see Org. Syn. 816 (1958)) using the appropriate starting materials. Furthermore, it can be elaborated to esters such as 2 using a transformation known to those skilled in the art as a Horner-Wadsworth-Emmons reaction (R and R′=alkyl) followed by hydrogenation using a transition metal catalyst such as palladium, platinum or rhodium in a solvent such as methanol. Alternatively, the transformation of 1 to 2 may be accomplished as described in U.S. Pat. No. 2,800,482. Compounds 3 can be prepared either by: 1) Addition of an excess of the appropriate organometallic reagent R1M (M=Li or Mg) in an etheral solvent such as tetrahydrofuran yielding compounds 3 in which R1=R2 or 2) By appropriately controlling the reaction conditions (or by transforming the ester 2 into a so called Weinreb amide 9—see SCHEME 2) the intermediate ketones 10 may be isolated and subsequently treated with R2M (M=Li or Mg) to form compounds 3 in which R1≠R2.

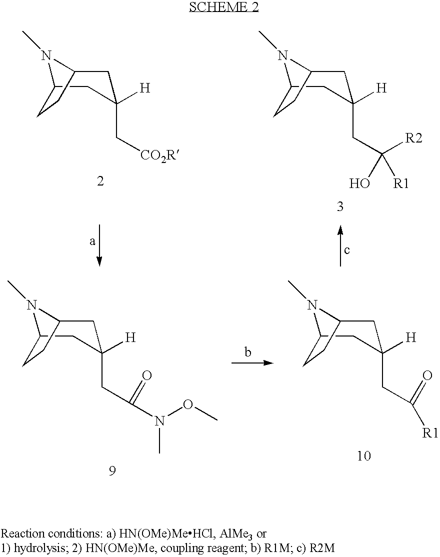

Alcohols 3 may then be treated with a reagent MeX (X=halide or sulfonate) to form the quarternary ammonium salts 6. Alternatively, 3 can undergo a process known to those skilled in the art as dehydration yielding the alkenes 4, which subsequently can be transformed to the corresponding quarternary ammonium salts 7 as described above. The alkenes 4 can also undergo hydrogenation as described previously to form the alkanes 5, which similarly can react with MeX to give the quarternary ammonium salts 8. Alternatively, the dehydration and hydrogenation steps may also be performed on the quarternary ammonium salts 6 and 7, respectively.

### SYNTHETIC EXAMPLES

The invention will now be described by reference to the following Examples which are merely illustrative and are not to be construed as a limitation of the scope of the present invention. All temperatures are given in ° C. Thin layer chromatography (t.l.c.) was carried out on silica, and column chromatography on silica (Flash column chromatography using Merck 9385 unless stated otherwise). LC/MS was conducted under the following conditions:

Column: 3.3 cm×4.6 mm ID, 3 um ABZ+PLUS
Flow Rate: 3 ml/min

### Injection Volume: 5 μl

### Temp: RT

Solvents: A: 0.1% aqueous Formic Acid+10 mMolar Ammonium Acetate.
B: 95% Acetonitrile+0.05% Formic Acid

| | Time | A % | B % |
|---|---|---|---|
| Gradient: | 0.00 | 100 | 0 |
| | 0.70 | 100 | 0 |
| | 4.20 | 0 | 100 |
| | 5.30 | 0 | 100 |
| | 5.50 | 100 | 0 |

### General Procedures:

### A. Grignard Reaction

The Grignard reagent (8 eq), which was prepared according to standard method or commercial available, was cooled to 0° C. with ice bath. The tropane ester (1 eq) in anhydrous tetrahedron furan (4 ml/mmol) was added dropwise. After warming to room temperature and stirring at room temperature for half an hour, the reaction mixture was heated to reflux for 2 hours. The reaction mixture was quenched with aqueous saturated ammonium chloride and extracted the aqueous phase with ethyl acetate. The organic phase was concentrated and purified by reverse-phase HPLC to afford product.

### B. Dehydration

The alcohol compounds were converted to alkene ones by one of the following methods.

1. A mixture of 1 g of the alcohol, 2 g of oxalic acid, and 3 ml of water is heated at reflux temperature for 2 hours. The cooled mixture is made alkaline with 10% NaOH and the product is removed by extraction with three portions of ether. Evaporation of the ether gives the desired alkene product.

2. A mixture of 1 g of the alcohol and 5 ml of 6N aqueous HCl is heated at reflux temperature for 1 hour. The cooled mixture is made alkaline with 10% NaOH and the product is removed by extraction with three portions of ether. Evaporation of the ether gives the desired alkene product.

3. A mixture of the alcohol and Amberlyst-15 (wet) resin (0.5 eq by weight) was stirred in 5:1 acetonitrile:water, and heated to 40° C. for 18 hours. The reaction is cooled and filtered. Evaporation gives the desired alkene product.

### C. Hydrogenation

The alkene was dissolved in methanol with 10% Palladium on carbon (0.5% mmol). The reaction mixture was stirred at room temperature with a hydrogen balloon for 2 hours. LCMS showed no starting material left. The reaction mixture was filtered with a pad of celite. The filtrate was concentrated to give alkane product. No purification was needed for this step.

### D. Quaternarization

The tertiary amine intermediates may be converted to quarternary ammonium slats using one of the following methods:

1. Tertiary amine (1 eq) and alkyl/aryl halide (20 eq) were dissolved in dichloromethane/acetonitrile (2:1) at room temperature. The resulting mixture was stirred at room temperature for 12 hours. The reaction mixture was concentrated to afford product without. In some cases, the residue was purified by reverse-phase HPLC (without TFA).

2. Tertiary amine (1 eq) was dissolved in acetone with bromomethane (20 eq) at room temperature. The resulting solution was stirred at room temperature for 12 hours. The reaction mixture was filtered off and washed with cold ether to give the quaternary salts as white solid.

3. Tertiary amine (1 eq) and aryl bromide (1 eq) were dissolved in chloroform/acetonitrile (3:2). The resulting solution was refluxed for 12 hours. The solvent was evaporated and the residue was purified by reverse phase preparative HPLC to afford product.

### Intermediate 1

### (3-Endo)-(8-methyl-8-aza-bicyclo[3.2.1]oct-3-yl)-acetic acid methyl ester

Neat trimethylphosphonacetate (19.6 ml, 0.121 mol) was added to a slurry of sodium hydride (95%, 3.15 g, 0.125 mol) in THF (150 ml) at ca −45° C. The resulting mixture was stirred between −45° C. and −35° C. for one hour. A solution of tropinone (15 g, 0.108 mol) in THF (100 ml) was added and the resulting mixture was stirred from −30° C. to room temperature over 2 hours. The reaction mixture was heated at reflux for 24 hours. After cooling to room temperature, the reaction mixture was quenched with water (50 ml), and then concentrated under vacuum to give a residue which was partitioned between 2M HCl (150 ml) and ether (400 ml). The aqueous phases were separated, washed with ether (2×200 ml) then basified to pH 12 with 2.5 M NaOH (ca 150 ml). The aqueous residue was then extracted with ethyl acetate (4×100 ml). The combined organics were dried over MgSO₄ and concentrated to give a crude oil (16 g, 76%).

NMR showed the desired product and about 5% of the SM. No trace of the endo alkene 2 was detected. LC/MS: 1.06 min (100%) corresponding to (M+H): 196.

10% Pd/C (1 g) was added to the above crude oil diluted in MeOH (400 ml). The resulting reaction mixture was allowed to hydrogenate at room temperature under 40 to 56 psi. After ca 43 hours no H₂ intake was observed. After filtration of the catalyst over Celite, the solvent was evaporated under vacuum to give a crude oil which was purified by distillation to give 11.2 g of colorless oil (69%) b.p. 122-125° C. NMR showed only the desired product. Less than 10% of the endo product might be present.

### Intermediate 2

### (3-Endo)-(8-methyl-8-aza-bicyclo[3.2.1]oct-3-yl)-acetic acid ethyl ester

### Ethyl cyano 3-tropaneacetate

A mixture of tropinone (13.9 g, 0.1 mol), ethyl cyanoacetate (11.3 g, 0.1 mol), ammonium acetate (1.6 g, 0.021 mol), acetic acid (7.3 g, 0.12 mol) and 10% Pd/C (0.6 g) in absolute ethanol (20 ml) was hydrogenated 60 p.s.i. at 50° C. for 18 h. After filtering off the catalyst, the filtrate was evaporated in vacuo. The amber oily residue is dissolved in dilute hydrochloric acid (1N, 200 ml) and the solution is extrated with ether (200 ml). The acid solution was neutralized and saturated with K₂CO₃ and the product removed by extraction with ether (6×200 ml). Distillation of the ether solution gave the desired ethyl cyano 3-tropaneacetate as a yellow oil, 8.0 g (34%) b.p. 139-140° C. (2 mm).

### Ethyl 3-tropaneacetate

A solution of 5.6 g of ethyl cyano-3-tropaneacetate in 25 ml of 37% hydrochloric acid was heated at relux for 13 h. The solution was evaporated in vacuo and the residue dried by successive addition and removal by distillation of absolute ethanol. The crude was esterified by allowing its solution in 40 ml of absolute ethanol saturated with hydrogen chloride to stand overnight at room temperature. Most of the alcohol was removed in vacuo. Then cold 5N NaOH solution (20 ml) was added to the residue and the product was extrated with ether (6×50 ml). Removal of ether gave the desired product as a pale yellow oil. Yield: 5.0 g (100%)

### Intermediate 3

### (3-Endo)-1,1-Bis-(3-thienyl)-2-(8-methyl-8-azabicyclo[3.2.1]oct-3-yl)ethanol

A solution of 3-bromothiophene (1.93 g, 11.8 mmol) in ether (6 ml) was cooled to −70° C. and added with stirring to a solution of n-butyl lithium (2.5 M in hexane, 4.8 ml) at −70° C. under Ar. The reaction mixture was stirred at −70° C. for 30 min. (reference: J.C.S. Perkin Trans. I. 1984, 223). (3-endo)-(8-methyl-8-aza-bicyclo[3.2.1]oct-3-yl)-acetic acid ethyl ester (1.00 g, 4.74 mmol) in ether was added via canula, and the solution was kept stirring at −70° C. for 1 hour. Water (10 ml) was added and the reaction mixture allowed warmed up to room temperature. The reaction mixture was then extracted with ether and washed with saturated NaCl. The ether layer was dried over Na2SO4 and evaporated to give crude product, which was purified by reverse-phase HPLC to afford about 460 mg of white solid (29%). LC/MS: (M+H): 334.

### Intermediate 4

### (3-Endo-3-(2,2-Bis-(3-thienyl)ethenyl)-8-methyl-8-azabicyclo[3.2.1]octane

The title compound was prepared from (3-endo-1,1-Bis-(3-thienyl)-2-(8-methyl-8-azabicyclo[3.2.1]oct-3-yl)ethanol (420 mg, 1.18 mmol) according to the general method B1 in 88% yield (420 mg). LC/MS: (M+H): 316.

### Intermediate 5

### (3-Endo)-3-(2,2-Bis-(3-thienyl)ethyl)-8-methyl-8-azabicyclo[3.2.1]octane

The title compound was prepared from (3-endo)-3-(2,2-Bis-(3-thienyl)ethenyl)-8-methyl-8-azabicyclo[3.2.1]octane (360 mg, 1.68 mmol) according to the general method C in 69% yield (250 mg). LC/MS: (M+H): 318.

### Intermediate 6

### (3-Endo)-1,1-bis(3,4-difluorophenyl)-2-(8-methyl-8-azabicyclo[3.2.1]oct-3-yl)ethanol

The title compound was prepared from (3-endo)-(8-methyl-8-aza-bicyclo[3.2.1]oct-3-yl)-acetic acid methyl ester (744 mg, 3.78 mmol) and 3,4-difluorophenyl magnesium bromide (0.5 M in THF, 48 ml, 24 mmol) according to the general method A in 54% yield (802 mg). LC/MS: (M+H): 394.

### Intermediate 7

### (3-Endo)3-[2,2-bis(3,4-difluorophenyl)ethenyl]-8-methyl-8-azabicyclo[3.2.1]octane

The title compound was prepared from (3-endo)-1,1-bis(3,4-difluorophenyl)-2-(8-methyl-8-azabicyclo[3.2.1]oct-3-yl)ethanol (430 mg, 1.09 mmol) according to the general method B1 in 92% yield (376 mg). LC/MS: (M+H): 376.

### Intermediate 8

### (3-Endo)3-[2,2-bis(3,4-difluorophenyl)ethyl]-8-methyl-8-azabicyclo[3.2.1]octane

The title compound was prepared from (3-endo)-3-[2,2-bis(3,4-difluorophenyl)ethenyl]-8-methyl-8-azabicyclo[3.2.1]octane (82 mg, 0.22 mmol) according to the general method C in 91% yield (75 mg). LC/MS: (M+H): 378.

### Intermediate 9

### (3-Endo)-1,1-bis(3,5-difluorophenyl)-2-(8-methyl-8-azabicyclo[3.2.1]oct-3-yl)ethanol

The title compound was prepared from (3-endo)-(8-methyl-8-aza-bicyclo[3.2.1]oct-3-yl)-acetic acid methyl ester (750 mg, 3.81 mmol) and 3,5-difluorophenyl magnesium bromide (0.5 M in THF, 50 ml, 25 mmol) according to the general method A in 19% yield (284 mg). LC/MS: (M+H): 394.

### Intermediate 10

### (3-Endo)3-[2,2-bis(5-difluorophenyl)ethenyl]-8-methyl-8-azabicyclo[3.2.1]octane

The title compound was prepared from (3-endo)-1,1-bis(3,5-difluorophenyl)-2-(8-methyl-8-azabicyclo[3.2.1]oct-3-yl)ethanol (270 mg, 0.68 mmol) according to the general method B1 in 74% yield (189 mg). LC/MS: 1.87 min, (M+H): 376.

### Intermediate 11

### (3-Endo)3-[2,2-bis(3,5-difluorophenyl)ethyl]-8-methyl-8-azabicyclo[3.2.1]octane

The title compound was prepared from (3-endo)-3-[2,2-bis(3,5-difluorophenyl)ethenyl]-8-methyl-8-azabicyclo[3.2.1]octane (50 mg, 0.133 mmol) according to the general method C in 80% yield (40 mg). LC/MS: (M+H): 378.

### Intermediate 12

### (3-Endo)-1,1-bis[5-fluoro-2-(methyloxy)phenyl]-2-(8-methyl-8-azabicyclo[3.2.1]oct-3-yl)ethanol

The title compound was prepared from (3-endo)-(8-methyl-8-aza-bicyclo[3.2.1]oct-3-yl)-acetic acid methyl ester (750 mg, 3.81 mmol) and 2-methoxy-5-fluorophenyl magnesium bromide (0.5 M in THF, 50 ml, 25 mmol) according to the general method A in 53% yield (842 mg). LC/MS: (M+H): 418.

### Intermediate 13

### (3-Endo)-3-{2,2-bis[5-fluoro-2-(methyloxy)phenyl]ethenyl}-8-methyl-8-azabicyclo[3.2.1]octane

The title compound was prepared from (3-endo)-1,1-bis[5-fluoro-2-(methyloxy)phenyl]-2-(8-methyl-8-azabicyclo[3.2.1]oct-3-yl)ethanol (195 mg, 0.68 mmol) according to the general method B1 in 46% yield (124 mg)

LC/MS: (M+H): 399.

### Intermediate 14

### (3-Endo)-1,1-bis(3-fluoro-2-methylphenyl)-2-(8-methyl-8-azabicyclo[3.2.1]oct-3-yl)ethanol

The title compound was prepared from (3-endo)-(8-methyl-8-aza-bicyclo[3.2.1]oct-3-yl)-acetic acid methyl ester (985 mg, 5.0 mmol) and 5-fluoro-2-methylphenyl magnesium bromide (0.5 M in THF, 60 ml, 30 mmol) according to the general method A in 11% yield (229 mg). LC/MS: (M+H): 418.

### Intermediate 17

### (3-Endo)-1,1-bis[5-fluoro-2-methylphenyl]-2-(8-methyl-8-azabicyclo[3.2.1]oct-3-yl)ethanol

The title compound was prepared from (3-endo)-(8-methyl-8-aza-bicyclo[3.2.1]oct-3-yl)-acetic acid methyl ester (985 mg, 5.0 mmol) and 5-fluoro-2-methylphenyl magnesium bromide (0.5 M in THF, 60 ml, 30 mmol) according to the general method A in 14% yield (292 mg). LC/MS: (M+H): 418.

### Intermediate 18

### (3-Endo)-3-[2,2-bis(5-fluoro-2-methylphenyl)ethenyl]-8-methyl-8-azabicyclo[3.2.1]octane

The title compound was prepared from (3-endo)-1,1-bis(5-fluoro-2-methylphenyl)-2-(8-methyl-8-azabicyclo[3.2.1]oct-3-yl)ethanol (140 mg, 0.36 mmol) according to the general method B2 in 98% yield (129 mg). LC/MS: (M+H): 368.

### Intermediate 19

### (3-Endo)-3-[2,2-bis(5-fluoro-2-methylphenyl)ethyl]-8-methyl-8-azabicyclo[3.2.1]octane

The title compound was prepared from (3-endo)-3-[2,2-bis(3-fluoro-2-methylphenyl)ethenyl]-8-methyl-8-azabicyclo[3.2.1]octane (80 mg, 0.22 mmol) according to the general method C in 78% yield (63 mg). LC/MS: (M+H): 370.

Intermediate 20

### (3-Endo)-1,1-dicyclohexyl-2-(8-methyl-8-azabicyclo[3.2.1]oct-3-yl)ethanol

The title compound was prepared from (3-endo)-(8-methyl-8-aza-bicyclo[3.2.1]oct-3-yl)-acetic acid methyl ester (400 mg, 2.03 mmol) and cyclohexyl magnesium bromide (0.5 M in ether, 30 ml, 15 mmol) according to the general method A in 15% yield (101 mg). LC/MS (M+H): 334.

### Intermediate 21

### (3-Endo)-1,1-dicyclopentyl-2-(8-methyl-8-azabicyclo[3.2.1]oct-3-yl)ethanol

The title compound was prepared from (3-endo)-(8-Methyl-8-aza-bicyclo[3.2.1]oct-3-yl)-acetic acid methyl ester (1.0 g, 5.03 mmol) and cyclopentyl magnesium bromide (2M in ether, 40 ml, 80 mmol) according to the general method A in 10% yield (153 mg). LC/MS: (M+H): 306.

### Intermediate 22

### (3-Endo)-1,3-bis(2-fluorophenyl)-2-[(8-methyl-8-azabicyclo[3.2.1]oct-3-yl)methyl]-2-propanol

The title compound was prepared from (3-endo)-(8-Methyl-8-aza-bicyclo[3.2.1]oct-3-yl)-acetic acid methyl ester (1.0 g, 5.03 mmol) cyclopentyl magnesium bromide (0.5 M in THF, 40 ml) according to the general method A in 25% yield (487 mg). LC/MS: (M+H): 386.

### Intermediate 23

### 2-[(3-Endo-8-methyl-8-azabicyclo[3.2.1]oct-3-yl]-1,1-di-2-pyridinylethanol

A solution of 2-bromopyridine (1.86 g, 11.8 mmol) in ether (6 ml) was cooled to −20° C. and added with stirring to a solution of n-butyl lithium (2.5 M in hexane, 4.8 ml) at −20° C. under Ar. The reaction mixture was stirred at −20° C. for 15 min. (reference: JACS 1951, 1788). (3-Endo)-(8-methyl-8-aza-bicyclo[3.2.1]oct-3-yl)-acetic acid ethyl ester (0.80 g, 3.77 mmol) in ether was added via canula, and the solution was kept stirring at −20° C. for 30 min. Water (10 ml) was added and the reaction mixture allowed warmed up to room temperature. The reaction mixture was then extracted with ether and washed with saturated NaCl. The ether layer was dried over Na₂SO₄ and evaporated to give crude product, which was purified by reverse-phase HPLC to afford about 200 mg of oily byproduct (10%). LC/MS: (M+H): 334.

### Intermediate 25

### 2-((3-Endo)-8-Methyl-8-aza-bicyclo[3.2.1]oct-3-yl)-1,1-bis-(3-methyl-2-thienyl)-ethanol

The title compound was synthesized according to U.S. Pat. No. 2,800,482, from (Endo-8-methyl-8-aza-bicyclo[3.2.1]oct-3-yl)-acetic acid methyl ester (0.50 g, 2.54 mmol) and 2-bromo-3-methyl thiophene (1.0 g, 5.65 mmol) and butyl lithium (2M in pentane, 2.8 ml, 5.65 mmol). Crude compound was purified by flash chromatography on silica using 1.8% NH₄OH:8% MeOH:92.2% CH₂Cl₂, yielding 0.320 g (34%). LC/MS (M+H): 362.

### Example 1

### (3-Endo)-3-[2-Hydroxy-2,2-bis-(3-methyl-2-thienyl)-ethyl]-8,8-dimethyl-8-azonia-bicyclo[3.2.1]octane bromide

The title compound was synthesized from 2-((3-endo)-8-Methyl-8-aza-bicyclo[3.2.1]oct-3-yl)-1,1-bis-(3-methyl-2-thienyl))-ethanol (0.320 g, 0.885 mmol) and methyl bromide (2M in t-Butyl methyl ether 2.2 ml, 4.4 mmol) according to the general method D1 yielding 0.248 g (61%). LC/MS (M+H): 376.

### Intermediate 26

### 1,1-Bis-(3-methoxy-phenyl)-2-((3-endo)-8-methyl-8-aza-bicyclo[3.2.1]oct-3-yl))-ethanol

Prepared from (Endo-8-methyl-8-aza-bicyclo[3.2.1]oct-3-yl)-acetic acid methyl ester (0.50 g, 2.54 mmol) and 3-methoxy magnesium bromide (1.0M in THF, 22 ml, 22 mmol) according to general method A and purified on silica using 1.8% NH₄OH:8% MeOH:92.2% CH₂ as solvent system, yielding 0.69 g (71%). LC/MS (M+H): 382.

### Example 2

### (3-Endo)-3-[2-Hydroxy-2,2-bis-(3-methoxy-phenyl)-ethyl]-8,8-dimethyl-8-azonia-bicyclo[3.2.1]octane iodide

Title compound was synthesized from 1,1-Bis-(3-methoxy-phenyl)-2-((3-endo)-8-methyl-8-aza-bicyclo[3.2.1]oct-3-yl))-ethanol (0.54 g, 1.42 mmol) and methyl iodide (530 μl, 8.5 mmol) according to general method D1, yielding 0.72 g (97%). LC/MS (M+H): 396.

### Intermediate 27

### (3-Endo)-3-[2,2-Bis-(3-hydroxy-phenyl)-vinyl]-8,8-dimethyl-8-azonia-bicyclo[3.2.1]octane bromide

(3-Endo)-3-[2-Hydroxy-2,2-bis-(3-methoxy-phenyl)-ethyl]-8,8-dimethyl-8-azonia-bicyclo[3.2.1]octane iodide, was dissolved in 6 ml of a 30% hydrogen bromide solution in acetic acid. It was heated to 70° C. for 9 hours and at room temperature for 12 hours. The solution was them concentrated and purified on reversed phase HPLC yielding 0.90 g of title compound. LC/MS (M+H): 350.

### Example 3

### (3-Endo)-3-[2,2-Bis-(3-hydroxy-phenyl)-ethyl]-8,8-dimethyl-8-azonia-bicyclo[3.2.1]octane bromide

(3-Endo)-3-[2,2-Bis-(3-hydroxy-phenyl)-vinyl]-8,8-dimethyl-8-azonia-bicyclo[3.2.1]octane bromide was dissolved in 15 ml methanol with 10% palladium on carbon (cat.), and placed on a PARR hydrogenator apparatus at 50 psi for two days after which it was filtered, concentrated yielding 0.030 g of title compound. LC/MS (M+H): 352.

### Intermediate 28

### 2-((3-Endo)-8-Methyl-8-aza-bicyclo[3.2.1]oct-3-yl)-1,1-bis-(4-methyl-3-thienyl)-ethanol

The title compound was synthesized according to U.S. Pat. No. 2,800,482, from ((3-endo)-8-methyl-8-aza-bicyclo[3.2.1]oct-3-yl)-acetic acid methyl ester (0.50 g, 2.54 mmol) and 3-bromo-4-methyl thiophene (1.0 g, 5.65 mmol) and butyl lithium (2 M in pentane, 2.8 ml, 5.65 mmol). Crude compound was purified by flash chromatography on silica using 1.8% NH₄OH:8% MeOH:92.2% CH₂Cl₂, yielding 0.242 g. LC/MS (M+H): 362.

### Intermediate 29

### 2-((3-Endo)-8-Methyl-8-aza-bicyclo[3.2.1]oct-3-yl)-1,1-bis-(5-methyl-2-thienyl))-ethanol

The title compound was synthesized according to U.S. Pat. No. 2,800,482, from ((3-endo)-8-methyl-8-aza-bicyclo[3.2.1]oct-3-yl)-acetic acid methyl ester (0.50, 2.54 mmol) and 2-bromo-5-methyl thiophene (1.0 g, 5.65 mmol) and butyl lithium (2M in pentane, 2.8 ml, 5.65 mmol). Crude compound was purified by flash chromatography on silica using 1.8% NH₄OH:8% MeOH:92.2% CH₂Cl₂, yielding 0.494 g. LC/MS (M+H): 362.

### Example 4

### (3-Endo)-3-[2-Hydroxy-2,2-bis-(4-methyl-3-thienyl)-ethyl]-8,8-dimethyl-8-azonia-bicyclo[3.2.1]octane bromide

The title compound was synthesized from 2-((3-endo)-8-Methyl-8-aza-bicyclo[3.2.1]oct-3-yl)-1,1-bis-(4-methyl-3-thienyl))-ethanol (0.120 g, 0.33 mmol) and methyl bromide (2M in t-Butyl methyl ether 0.83 ml, 1.65 mmol) according to the general method D1 yielding 0.048 g (31%). LC/MS (M+H): 376.

### Example 5

### (3-Endo)-3-[2-Hydroxy-2,2-bis-(5-methyl-2-thienyl)-ethyl]-8,8-dimethyl-8-azonia-bicyclo[3.2.1]octane bromide

The title compound was synthesized from 2-((3-endo)-8-Methyl-8-aza-bicyclo[3.2.1]oct-3-yl)-1,1-bis-(5-methyl-2-thienyl))-ethanol (0.247 g, 0.68 mmol) and methyl bromide (2M in t-Butyl methyl ether 1.7 ml, 3.4 mmol) according to the general method D1 yielding 0.143 g (46%). LC/MS (M+H): 376.

### Intermediate 33

### 1,1-Bis-(5-chloro-2-thienyl)-2-((3-endo)-8-methyl-8-aza-bicyclo[3.2.1]oct-3-yl)-ethanol

The title compound was synthesized according to U.S. Pat. No. 2,800,482, from ((3-endo)8-methyl-8-aza-bicyclo[3.2.1]oct-3-yl))-acetic acid methyl ester (0.338 g, 1.72 mmol) and 2-bromo-5-chloro thiophene (395 μl, 3.6 mmol) and butyl lithium (2M in pentane, 1.8 ml, 3.6 mmol), yielding 0.470 g. Further purification was not performed. LC/MS (M+H): 402.

### Example 6

### (3-Endo)-3-[2,2-Bis-(5-chloro-2-thienyl)-2-hydroxy-ethyl]-8,8-dimethyl-8-azonia-bicyclo[3.2.1]octane bromide

The title compound was synthesized from 1,1-Bis-(5-chloro-2-thienyl)-2-((3-endo)-8-methyl-8-aza-bicyclo[3.2.1]oct-3-yl))-ethanol (0.220 g, 0.55 mmol) and methyl bromide (2M in t-Butyl methyl ether 1.3 ml, 2.7 mmol) according to the general method D3. It was purified by reversed phase HPLC yielding 0.11 g (40%). LC/MS (M+H): 416.

### Intermediate 35

### 1,1-Bis-[5-(1,1-difluoro-methyl)-2-thienyl]-2-((3-endo)-8-methyl-8-aza-bicyclo[3.2.1]oct-3-yl)-ethanol

The title compound was synthesized according to U.S. Pat. No. 2,800,482, from ((3-endo)-8-methyl-8-aza-bicyclo[3.2.1]oct-3-yl)-acetic acid methyl ester (0.242 g, 1.23 mmol) and 2-bromo-5-(1,1-difluoro-methyl)-thiophene (prepared according to JOC 64, 7048, (1999), 0.544 g, 2.58 mmol) and butyl lithium (2M in pentane, 1.3 ml, 5.65 mmol). Crude compound was purified by flash chromatography on silica using 1.8% NH₄OH:8% MeOH:92.2% CH₂Cl₂, yielding 0.380 g. LC/MS (M+H): 434.

### Example 7

### (3-Endo)-3-{2,2-Bis-[5-(1,1-difluoro-methyl)-2-thienyl]-2-hydroxy-ethyl}-8,8-dimethyl-8-azonia-bicyclo[3.2.1]octane bromide

The title compound was synthesized from 1,1-Bis-[5-(1,1-difluoro-methyl)-2-thienyl]-2-((3-endo)-8-methyl-8-aza-bicyclo[3.2.1]oct-3-yl)-ethanol (0.150 g, 0.346 mmol) and methyl bromide (2M in t-Butyl methyl ether 0.86 ml, 1.73 mmol) according to the general method D1. It was purified by reversed phase HPLC yielding 0.107 g (61%). LC/MS (M+H): 448.

### Example 8

### (3-Endo)-1,1-Bis-(3-thienyl)-2-(8,8-dimethyl-8-azoniabicyclo[3.2.1]oct-3-yl)ethanol iodide

The title compound was prepared from (3-endo)-1,1-Bis-(3-thienyl)-2-(8-methyl-8-azabicyclo[3.2.1]oct-3-yl)ethanol (100 mg, 0.30 mmol) and iodoomethane (890 mg, 6 mmol) according to the general method D1 in 91% yield (117 mg). LC/MS: (M+H): 348.

### Example 9

### (3-Endo)-3-(2,2-Bis-(3-thienyl)ethyl)-8,8-dimethyl-8-azoniabicyclo[3.2.1]octane iodide

The title compound was prepared from (3-endo)-3-(2,2-Bis-(3-thienyl)ethyl)-8-methyl-8-azabicyclo[3.2.1]octane (50 mg, 0.16 mmol) and iodoomethane (466 mg, 3.2 mmol) according to the general method D1 in 60% yield (340 mg). LC/MS: (M+H): 332.

### Example 10

### (3-Endo)-1,1-bis(3,4-difluorophenyl)-2-(8,8-dimethyl-8-azoniabicyclo[3.2.1]oct-3-yl)ethanol bromide

The title compound was prepared from (3-endo)-1,1-bis(3,5-difluorophenyl)-2-(8-methyl-8-aza-bicyclo[3.2.1]oct-3-yl)ethanol (100 mg, 0.254 mmol) and bromomethane (2.5 ml of 2M in tert-butyl ether, 5.0 mmol) according to the general method D2 in 71% yield (88 mg). LC/MS: (M+H): 408.

### Example 11

### (3-Endo)-3-[2,2-bis(3,4-difluorophenyl)ethyl]-8,8-dimethyl-8-azoniabicyclo[3.2.1]octane bromide

The title compound was prepared from (3-endo)-3-[2,2-bis(3,4-difluorophenyl)ethyl]-8-methyl-8-azabicyclo[3.2.1]octane (42 mg, 0.11 mmol) and bromomethane (1.1 ml, 2M in tert-butyl ether, 2.2 mmol) according to the general method D2 in 76% yield (139 mg). LC/MS: (M+H): 392.

### Example 12

### (3-Endo)-1,1-bis(3,5-difluorophenyl)-2-(8,8-dimethyl-8-azoniabicyclo[3.2.1]oct-3-yl)ethanol bromide

The title compound was prepared from (3-endo)-1,1-bis(3,5-difluorophenyl)-2-(8-methyl-8-aza bicyclo[3.2.1]oct-3-yl)ethanol (36 mg, 0.092 mmol) and bromomethane (0.9 ml, 2M in tert-butyl ether, 1.8 mmol) according to the general method D2 in 79% yield (35 mg). LC/MS: (M+H): 408.

### Example 13

### (3-Endo)3-[2,2-bis(3,5-difluorophenyl)ethyl]-8,8-dimethyl-8-azoniabicyclo[3.2.1]octane bromide

The title compound was prepared from (3-endo)-3-[2,2-bis(3,5-difluorophenyl)ethyl]-8-methyl-8-azabicyclo[3.2.1]octane (50 mg, 0.133 mmol) and bromomethane (1.3 ml, 2M in tert-butyl ether, 52.6 mmol) according to the general method D2 in 34% yield (21 mg). LC/MS: (M+H): 392.

### Example 14

### (3-Endo)-1,1-bis[5-fluoro-2-(methyloxy)phenyl]-2-(8,8-dimethyl-8-azoniabicyclo[3.2.1]oct-3-yl)ethanol bromide

The title compound was prepared from (3-endo)-1,1-bis[5-fluoro-2-(methyloxy)phenyl]-2-(8-methyl-8-azabicyclo[3.2.1]oct-3-yl)ethanol (100 mg, 0.240 mmol) and bromomethane (2.4 ml, 2M in tert-butyl ether, 4.8 mmol) according to the general method D2 in 84% yield (103 mg). LC/MS: (M+H): 432.

### Example 15

### (3-Endo)-1,1-bis(3-fluoro-2-methylphenyl)-2-(8,8-dimethyl-8-azoniabicyclo[3.2.1]oct-3-yl)ethanol bromide

The title compound was prepared from (3-endo)-1,1-bis(5-fluoro-2-methylphenyl)-2-(8-methyl-8-azabicyclo[3.2.1]oct-3-yl)ethanol (50 mg, 0.13 mmol) and bromomethane (1.3 ml, 2M in tert-butyl ether, 2.6 mmol) according to the general method D2 in 72% yield (45 mg). LC/MS: (M+H): 400.

### Example 16

### (3-Endo)-1,1-bis(5-fluoro-2-methylphenyl)-2-(8,8-dimethyl-8-azoniabicyclo[3.2.1]oct-3-yl)ethanol bromide

The title compound was prepared from (3-endo)-1,1-bis(5-fluoro-2-methylphenyl)-2-(8-methyl-8-azabicyclo[3.2.1]oct-3-yl)ethanol (50 mg, 0.13 mmol) and bromomethane (1.3 ml, 2M in tert-butyl ether, 2.6 mmol) according to the general method D2 in 84% yield (52 mg). LC/MS: (M+H): 400.

### Example 17

### (3-Endo)-3-[2,2-bis(5-fluoro-2-methylphenyl)ethyl]-8,8-dimethyl-8-azoniabicyclo[3.2.1]octane iodide

The title compound was prepared from (3-endo)-3-[2,2-bis(5-fluoro-2-methylphenyl)ethyl]-8-methyl-8-azabicyclo[3.2.1]octa (40 mg, 0.11 mmol) and iodoomethane (323 mg, 2.2 mmol) according to the general method D1 in 27% yield (14 mg). LC/MS: (M+H): 384.

### Example 18

### (3-Endo)-1,1-dicyclohexyl-2-(8,8-dimethyl-8-azoniabicyclo[3.2.1]oct-3-yl)ethanol bromide

The title compound was prepared from (3-endo)-1,1-di-dicyclohexyl-2-(8-methyl-8-azabicyclo[3.2.1]oct-3-yl)ethanol (65 mg, 0.195 mmol) and bromomethane (414 mg, 3.8 mmol) according to the general method D2 in 55% yield (66 mg). LC/MS: (M+H): 348.

### Example 19

### (3-Endo)-1,1-dicyclopentyl-2-(8,8-dimethyl-8-azoniabicyclo[3.2.1]oct-3-yl)ethanol bromide

The title compound was prepared from (3-endo)-1,1-di-dicyclopentyl-2-(8-methyl-8-azabicyclo[3.2.1]oct-3-yl)ethanol (120 mg, 0.39 mmol) and bromomethane (850 mg, 7.8 mmol) according to the general method D2 in 78% yield (100 mg). LC/MS: (M+H): 320.

### Example 20

### (3-Endo)-1,3-bis(2-fluorophenyl)-2-[(8,8-dimethyl-8-azoniabicyclo[3.2.1]oct-3-yl)methyl]-2-propanol bromide

The title compound was prepared from (3-endo)-1,3-bis(2-fluorophenyl)-2-[(8-methyl-8-azabicyclo[3.2.1]oct-3-yl)methyl]-2-propanol (55 mg, 0.143 mmol) and bromomethane (312 mg, 2.86 mmol) according to the general method D2 in 73% yield (50 mg). LC/MS: (M+H): 400.

### Example 21

### 2-[(3-(3-Endo))-8,8-dimethyl-8-azoniabicyclo[3.2.1]oct-3-yl]-1,1-di-2-pyridinylethanol iodide

The title compound was prepared from 2-[((3-endo))-8-methyl-8-azabicyclo[3.2.1]oct-3-yl]-1,1-di-2-pyridinylethanol (50 mg, 0.155 mmol) and iodomethane (28 mg, 0.19 mmol) according to the general method D1 in 60% yield (60 mg). LC/MS: (M+H): 338.

### Intermediate 38

### (3-Endo)-1,1-Bis-(4-chloro-phenyl)-2-(8-methyl-8-aza-bicyclo[3.2.1]oct-3-yl)-ethanol

The title compound was prepared from (3-endo)-(8-Methyl-8-aza-bicyclo[3.2.1]oct-3-yl)-acetic acid ethyl ester (600 mg, 2.85 mmol) and 4-chlorophenyl magnesium bromide (1 M in THF, 20 mL, 20 mmol) according to the general method A (554 mg) in 50% yield. LC/MS: (M+H): 390.

### Intermediate 39

### (3-Endo)-1,1-Bis-(3-chloro-phenyl)-2-(8-methyl-8-aza-bicyclo[3.2.1]oct-3-yl)-ethanol

The title compound was prepared from (3-endo)-(8-Methyl-8-aza-bicyclo[3.2.1]oct-3-yl)-acetic acid ethyl ester (800 mg, 4.06 mmol), magnesium (1.18 g, 48.7 mmol) and 3-chlorophenyl bromide (7.77 g, 40.6 mmol) according to the general method A (1.00 g) in 63.3% yield. LC/MS: (M+H): 390.

### Intermediate 40

### (3-Endo)-1,1-Bis-(4-fluoro-phenyl)-2-(8-methyl-8-aza-bicyclo[3.2.1]oct-3-yl)-ethanol

The title compound was prepared from (3-endo)-(8-Methyl-8-aza-bicyclo[3.2.1]oct-3-yl)-acetic acid ethyl ester (800 mg, 3.79 mmol) and 4-fluorophenyl magnesium bromide (1 M in THF, 31 mL, 30 mmol) according to the general method A (1.10 g) in 82% yield. LC/MS: (M+H): 358

### Intermediate 41

### (3-Endo)-1,1-Bis-(3-fluoro-phenyl)-2-(8-methyl-8-aza-bicyclo[3.2.1]oct-3-yl)-ethanol

The title compound was prepared from (3-endo)-(8-Methyl-8-aza-bicyclo[3.2.1]oct-3-yl)-acetic acid methyl ester (600 mg, 3.05 mmol), magnesium (888 mg, 36.5 mmol) and 3-fluorophenyl bromide (5.34 g, 30.5 mmol) according to the general method A (700 mg) in 64% yield. LC/MS: (M+H): 358

### Intermediate 43

### (3-Endo)-3-[2,2-Bis-(3-chloro-phenyl)-vinyl]-8-methyl-8-aza-bicyclo[3.2.1]octane

The title compound (400 mg) was prepared from (3-endo)-1,1-bis(3-chlorophenyl)-2-(8-methyl-8-azabicyclo[3.2.1]oct-3-yl)ethanol (500 mg, 1.28 mmol) according to the general method B1 in 84% yield. LC/MS: (M+H): 372

### Intermediate 44

### (3-Endo)-3-[2,2-Bis-(4-fluoro-phenyl)-vinyl]-8-methyl-8-aza-bicyclo[3.2.1]octane

The title compound (700 mg) was prepared from (3-endo)-1,1-bis(4-fluorophenyl)-2-(8-methyl-8-azabicyclo[3.2.1]oct-3-yl)ethanol (1000 mg, 2.80 mmol) according to the general method B1 in 74% yield. LC/MS: (M+H): 340

### Intermediate 45

### (3-Endo)-3-[2,2-Bis-(3-fluoro-phenyl)-vinyl]-8-methyl-8-aza-bicyclo[3.2.1]octane

The title compound (400 mg) was prepared from (3-endo)-1,1-bis(3-fluorophenyl)-2-(8-methyl-8-azabicyclo[3.2.1]oct-3-yl)ethanol (460 mg, 1.28 mmol) according to the general method B1 in 92% yield. LC/MS: (M+H): 340

### Example 22

### (3-Endo)-1,1-Bis-(4-fluoro-phenyl)-2-(8,8-dimethyl-8-azonia-bicyclo[3.2.1]oct-3-yl)-ethanol iodide

(3-Endo)-1,1-Bis-(4-fluoro-phenyl)-2-(8-methyl-8-aza-bicyclo[3.2.1]oct-3-yl)-ethanol (100 mg, 0.28 mmol), and 2.0 ml of methyl iodide (32.1 mmol) were stirred in 5 mL methanol at room temperature 12 hours. The reaction mixture was concentrated to give the title compound (103 mg, 99%). LC/MS: (M+H): 372.

### Intermediate 47

### (3-Endo)-3-[2,2-Bis-(4-fluoro-phenyl)-ethyl]-8-methyl-8-aza-bicyclo[3.2.1]octane

(3-Endo)-3-[2,2-Bis-(4-fluoro-phenyl)-vinyl]-8-methyl-8-aza-bicyclo[3.2.1]octane (400 mg, 1.18 mmol) was dissolved in methanol (10 mL) with 10% Palladium on carbon (50 mg, 0.5% mmol). The reaction mixture was stirred at room temperature with a hydrogen balloon for 2 hours. The reaction mixture was filtered with a pad of celite. The filtrate was concentrated to give title compound (270 mg, 67%). No purification was needed for this step. LC/MS: (M+H): 342.

### Example 23

### (3-Endo)-1,1-Bis-(4-chloro-phenyl)-2-(8,8-dimethyl-8-azonia-bicyclo[3.2.1]oct-3-yl)-ethanol iodide

(3-Endo)-1,1-Bis-(4-chloro-phenyl)-2-(8-methyl-8-aza-bicyclo[3.2.1]oct-3-yl)-ethanol (100 mg, 0.256 mmol), and 2.0 ml of methyl iodide (32.1 mmol) were stirred in a mixture of methylene chloride and acetylnitrile (10 ml, 2:1) at room temperature. LCMS showed no starting material left after the reaction was stirred for 12 hours. The reaction mixture was concentrated to give the title compound (74 mg, 72%). LC/MS: 1.96 min, (M+H): 406.

### Example 24

### (3-Endo)-3-[2,2-Bis-(4-fluoro-phenyl)-ethyl]-8,8-dimethyl-8-azonia-bicyclo[3.2.1]octane iodide

(3-Endo)-3-[2,2-Bis-(4-fluoro-phenyl)-ethyl]-8-methyl-8-aza-bicyclo[3.2.1]octane (200 mg, 0.58 mmol), and 2.0 mL of methyl iodide (32.1 mmol) were stirred in 5 mL methanol at room temperature 12 hours. The reaction mixture was concentrated to give the title compound (200 mg, 99%). LC/MS: (M+H): 356.

### Example 25

### (3-Endo)-1,1-Bis-(3-fluoro-phenyl)-2-(8,8-dimethyl-8-azonia-bicyclo[3.2.1]oct-3-yl)-ethanol iodide

(3-Endo)-1,1-Bis-(3-fluoro-phenyl)-2-(8-methyl-8-aza-bicyclo[3.2.1]oct-3-yl)-ethanol (100 mg, 0.28 mmol), and 0.5 mL of methyl iodide (8.1 mmol) were stirred in 5 ml methanol at room temperature for 12 hours. The reaction mixture was concentrated to give the title compound (90 mg, 86%). LC/MS: (M+H): 372

### Intermediate 48

### (3-Endo)-3-[2,2-Bis-(3-fluoro-phenyl)-ethyl]-8-methyl-8-aza-bicyclo[3.2.1]octane (3-Endo)-3-[2,2-Bis-(3-fluoro-phenyl)-vinyl]-8-methyl-8-aza-bicyclo[3.2.1]octane

(200 mg, 0.59 mmol) was dissolved in methanol (10 ml) with 10% Palladium on carbon (50 mg, 0.5% mmol). The reaction mixture was stirred at room temperature with a hydrogen balloon for 2 hours. LCMS showed no starting material left. The reaction mixture was filtered with a pad of celite. The filtrate was concentrated to give title compound (200 mg, 99%). No purification was needed for this step. LC/MS: (M+H): 342.

### Example 26

### (3-Endo)-1,1-Bis-(3-chloro-phenyl)-2-(8,8-dimethyl-8-azonia-bicyclo[3.2.1]oct-3-yl)-ethanol iodide

(3-Endo)-1,1-Bis-(3-chloro-phenyl)-2-(8-methyl-8-aza-bicyclo[3.2.1]oct-3-yl)-ethanol (200 mg, 0.53 mmol), and 1 ml of methyl iodide (16.1 mmol) were stirred in 5 ml methanol at room temperature. LCMS showed no starting material left after the reaction was stirred for 12 hours. The reaction mixture was concentrated to give the title compound (160 mg, 79%). LC/MS: (M+H): 404.

### Intermediate 49

### (3-Endo)-3-[2,2-Bis-(3-chloro-phenyl)-ethyl]-8-methyl-8-aza-bicyclo[3.2.1]octane and (3-Endo)-3-[2-(3-Chloro-phenyl)-2-phenyl-ethyl]-8-methyl-8-aza-bicyclo[3.2.1]octane

(3-Endo)-3-[2,2-Bis-(3-chloro-phenyl)-vinyl]-8-methyl-8-aza-bicyclo[3.2.1]octane (230 mg, 0.65 mmol) was dissolved in methanol (10 mL). 10% Palladium on carbon (50 mg, 0.5% mmol) was added. The reaction mixture was stirred at room temperature with a hydrogen balloon for 2 hours and filtered with a pad of celite. The filtrate was concentrated and purified by HPLC to afford title products (97 mg, 42% and 120 mg, 50%). LC/MS: (M+H): 374.

### Example 27

### (3-Endo)-3-[2,2-Bis-(3-chloro-phenyl)-ethyl]-8,8-dimethyl-8-azonia-bicyclo[3.2.1]octane iodide

(3-Endo)-3-[2,2-Bis-(3-chloro-phenyl)-ethyl]-8-methyl-8-aza-bicyclo[3.2.1]octane (97 mg, 0.26 mmol), and 0.5 mL of methyl iodide (8.1 mmol) were stirred in 5 mL methanol at room temperature for 12 hours. The reaction mixture was concentrated to give the title compound (50 mg, 48%). LC/MS: (M+H): 388.

### Example 28

### (3-Endo)-3-[2-(3-Chloro-phenyl)-2-phenyl-ethyl]-8,8-dimethyl-8-azonia-bicyclo[3.2.1]octane iodide

(3-Endo)-3-[2-(3-Chloro-phenyl)-2-phenyl-ethyl]-8-methyl-8-aza-bicyclo[3.2.1]octane (120 mg, 0.35 mmol), and 0.5 mL of methyl iodide (8.1 mmol) were stirred in 5 mL methanol at room temperature. LCMS showed no starting material left after the reaction was stirred for 12 hours. The reaction mixture was concentrated to give the title compound (90 mg, 72%). LC/MS: (M+H): 354.

### Example 29

### (3-Endo)-3-[2,2-Bis-(3-fluoro-phenyl)-ethyl]-8,8-dimethyl-8-azonia-bicyclo[3.2.1]octane iodide

(3-Endo)-3-[2,2-Bis-(3-fluoro-phenyl)-ethyl]-8-methyl-8-aza-bicyclo[3.2.1]octane (200 mg, 0.59 mmol), and 0.5 mL of methyl iodide (8.1 mmol) were stirred in 5 mL methanol at room temperature for 12 hours. The reaction mixture was concentrated to give the title compound (160 mg, 76%). LC/MS: (M+H): 356

### Intermediate 50

### (3-Endo)-2-(8-Methyl-8-aza-bicyclo[3.2.1]oct-3-yl)-1-phenyl-ethanone

To a slurry solution of (3-endo)-(8-Methyl-8-aza-bicyclo[3.2.1]oct-3-yl)-acetic acid methyl ester (1.97 g, 10 mmol) and N,O-Dimethylhydroxylamine hydrochloride (1.22 g, 12.5 mmol) in tetrahedrofuran (100 mL) at −5° C. was added 1 M phenylymagnesium bromide (30 mL, 30 mmol) over 10 minutes. The solution was stirred at −5° C. for 20 minutes before it was warmed up to room temperature overnight. The reaction was quenched with aqueous saturated ammonium chloride (40 mL) and extracted with ethyl acetate (100 ml×3). The combined organic phase was concentrated and purified by HPLC to afford title compound (1.9 g, 78%). LC/MS: (M+H): 244.

### Intermediate 51

### (3-Endo)-1-(2,3-Dichloro-phenyl)-2-(8-methyl-8-aza-bicyclo[3.2.1]oct-3-yl)-1-phenyl-ethanol

n-Butyllithium (2.5 M, 2.5 mL) was added to 1,2-dichlorobenzene (0.70 mL, 6.224 mmol) in 50 mL tetrahedrofuran at −78° C. dropwise over 10 minutes. The reaction mixture was stirred at −78 C for 2.5 hours before 2-((3-endo)-8-Methyl-8-aza-bicyclo[3.2.1]oct-3-yl))-1-phenyl-ethanone (500 mg, 2.06 mmol) was added. The solution was warmed up to room temperature, quenched with aqueous saturated ammonium chloride (15 mL) and extracted the aqueous phase with ethyl acetate (100 mL×3). The combined organic phase was concentrated and purified by HPLC to afford the product (480 mg, 59.7%). LC/MS: (M+H): 390.

### Intermediate 53

### (3-Endo)-1-(2,3-Fluoro-phenyl)-2-(8-methyl-8-aza-bicyclo[3.2.1]oct-3-yl)-1-phenyl-ethanol

n-Butyllithium (2.5 M, 5.0 mL) was added to 1,2-dichlorobenzene (1.2 ml, 12.36 mmol) in 20 mL tetrahedrofuran at −78 C dropwise over 10 minutes. The reaction mixture was stirred at −78 C for 2.0 hours before 2-((3-endo)-8-Methyl-8-aza-bicyclo[3.2.1]oct-3-yl))-1-phenyl-ethanone (500 mg, 2.06 mmol) was added. The solution was warmed up to room temperature, quenched with aqueous saturated ammonium chloride (15 ml) and extracted with ethyl acetate (3×100 ml). The combined organic phase was concentrated and purified by HPLC to afford the product (150 mg, 20.4%). LC/MS: (M+H): 358.

### Example 30

### (3-Endo)-1-(2,3-Fluoro-phenyl)-2-(8,8-dimethyl-8-azonia-bicyclo[3.2.1]oct-3-yl)-1-phenyl-ethanol iodide

(3-Endo)-1-(2,3-Fluoro-phenyl)-2-(8-methyl-8-aza-bicyclo[3.2.1]oct-3-yl)-1-phenyl-ethanol (20 mg, 0.073 mmol), and 1 mL of methyl iodide (16.1 mmol) were stirred in 5 mL methanol at room temperature for 12 hours. The reaction mixture was concentrated to give the title compound (23 mg, 85%). LC/MS: (M+H): 372.

### Example 31

### (3-Endo)-1-(2,3-Chloro-phenyl)-2-(8,8-dimethyl-8-azonia-bicyclo[3.2.1]oct-3-yl)-1-phenyl-ethanol iodide

(3-Endo)1-(2,3-Chloro-phenyl)-2-(8-methyl-8-aza-bicyclo[3.2.1]oct-3-yl)-1-phenyl-ethanol (50 mg, 0.128 mmol), and 1 mL of methyl iodide (16.1 mmol) were stirred in 5 ml methanol at room temperature for 12 hours. The reaction mixture was concentrated to give the title compound (40 mg, 77%). LC/MS: (M+H): 404.

### BIOLOGICAL EXAMPLES

The inhibitory effects of compounds at the M₃ mAChR of the present invention are determined by the following in vitro and in vivo functional assays:

### Analysis of Inhibition of Receptor Activation by Calcium Mobilization:

Stimulation of mAChRs expressed on CHO cells were analyzed by monitoring receptor-activated calcium mobilization as previously described (4). CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl₂, 1.1 mM MgCl₂, 11 mM glucose, 20 mM HEPES (pH 7.4)). 50 μl of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels (5). The emitted fluorescence from all 96 wells is measured simultaneously using a cooled CCD camera. Data points are collected every second. This data was then plotting and analyzed using GraphPad PRISM software.

### Methacholine-Induced Bronchoconstriction

Airway responsiveness to methacholine was determined in awake, unrestrained BalbC mice (n=6 each group). Barometric plethysmography was used to measure enhanced pause (Penh), a unitless measure that has been shown to correlate with the changes in airway resistance that occur during bronchial challenge with methacholine (2). Mice were pretreated with 50 μl of compound (0.003-10 μg/mouse) in 50 μl of vehicle (10% DMSO) intranasally, i.v., i.p. or p.o, and were then placed in the plethysmography chamber. Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis; gastrointestinal-tract disorders such as irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakisuria, neurogenic bladder, nocturnal enuresis, psychosomatic bladder, incontinence associated with bladder spasms or chronic cystitis, urinary urgency or pollakiuria, and motion sickness.

Methods of administering the present compounds will be readily apparent to the skilled artisan.

Dry powder compositions for topical delivery to the lung by inhalation may, for example, be presented in capsules and cartridges of for example gelatine, or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 20 μg-10 mg of the compound of formula (I) optionally in combination with another therapeutically active ingredient. Alternatively, the compound of the invention may be presented without excipients.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognised as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regime for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

A preferable means for applying the formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the pre-compression pump is that atomisation of the spray is ensured as it will not release the formulation until the threshold pressure for effectively atomising the spray has been achieved. Typically, the VP7 model may be used with a bottle capable of holding 10-50 ml of a formulation. Each spray will typically deliver 50-100 μl of such a formulation, therefore, the VP7 model is capable of providing at least 100 metered doses.

### Examples of Nasal Formulations

### Example 1

### Nasal Formulation Containing Active

A formulation for intranasal delivery was prepared with ingredients as follows:

| | to 100% |
|---|---|
| Active | 0.1% w/w |
| Polysorbate 80 | 0.025% w/w |
| Avicel RC591 | 1.5% w/w |
| Dextrose | 5.0% w/w |
| BKC | 0.015% w/w |
| EDTA | 0.015% w/w |
| water | to 100% |

in a total amount suitable for 120 actuations and the formulation was filled into a bottle fitted with a metering valve adapted to dispense 50 or 100 μl per actuation. The device was fitted into a nasal actuator (Valois).

### Example 2

### Nasal Formulation Containing Active

A formulation for intranasal delivery was prepared with ingredients as follows:

| | |
|---|---|
| Active | 0.005% w/w |
| Tyloxapol | 2% w/w |
| dextrose | 5% w/w |
| BKC | 0.015% w/w |
| EDTA | 0.015% w/w |
| water | to 100% |

in a total amount suitable for 120 actuations and the formulation was filled into a bottle (plastic or glass) fitted with a metering valve adapted to dispense 50 or 100 μl per actuation

The device was fitted into a nasal actuator (Valois, e.g. VP3, VP7 or VP7D)

### Example 3

### Nasal Formulation Containing Active

A formulation for intranasal delivery was prepared with ingredients as follows:

| | |
|---|---|
| active | 0.05% w/w |
| Triton X-100 | 5% w/w |
| Dextrose | 4% w/w |
| BKC | 0.015% w/w |
| EDTA | 0.015% w/w |
| water | to 100% |

in a total amount suitable for 120 actuations and the formulation was filled into a bottle fitted with a metering valve adapted to dispense 50 or 100 μl per actuation.

### Example 4

### Nasal Formulation Containing Active

A formulation for intranasal delivery was prepared with ingredients as follows:

| | |
|---|---|
| active | 0.05% w/w |
| Tyloxapol | 5% w/w |
| dextrose | 5% w/w |
| BKC | 0.015% w/w |
| EDTA | 0.015% w/w |
| water | to 100% |

in a total amount suitable for 120 actuations and the formulation was filled into a bottle fitted with a metering valve adapted to dispense 50 or 100 μl per actuation The device was fitted into a nasal actuator (Valois).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The patents and patent applications described in this application are herein incorporated by reference.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

1. A compound according to formula (I) 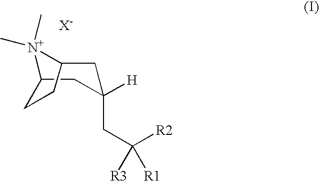
wherein:
R1 and R2 are, independently, selected from the group consisting of 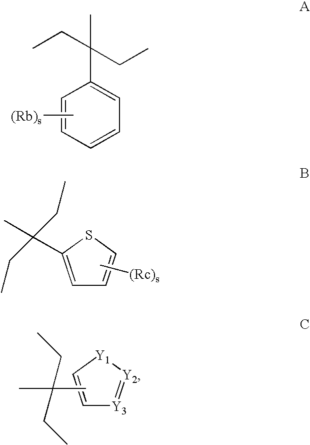
pyridyl, benzyl, pyrimidyl, thiazolyl, isothiazolyl and C₃₋₇cycloalkyl;
R3 is hydrogen or hydroxy;
R4 and R5 are, independently, selected from the group consisting of hydrogen and optionally substituted C₁₋₄alkyl;
Rb is, independently, selected from the group consisting of halogen, hydroxy, cyano, nitro, dihalomethyl, trihalomethyl and NR4R5;
Rc is, independently, selected from the group consisting of C₁₋₄alkyl, halogen, hydroxy, cyano, nitro, dihalomethyl, trihalomethyl and NR4R5;
X— is a physiologically acceptable anion,
Y1 is O or NR4;
Y2 and Y3 are, independently, selected from the group consisting of N and CH; or
s is an integer having a value of 1 to 3.

2. A compound according to claim 1 wherein the anion is selected from the group consisting of chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate.

3. A compound according to claim 1 selected from the group consisting of:
(3-Endo)-3-[2,2-Bis-(3-hydroxy-phenyl)-ethyl]-8,8-dimethyl-8-azonia-bicyclo[3.2.1]octane bromide;
(3-Endo)-3-[2,2-Bis-(3-chloro-phenyl)-ethyl]-8,8-dimethyl-8-azonia-bicyclo[3.2.1]octane iodide;
(3-Endo)-3-[2,2-Bis-(5-chloro-2-thienyl)-2-hydroxy-ethyl]-8,8-dimethyl-8-azonia-bicyclo[3.2.1]octane bromide;
(3-Endo)-1,1-Bis-(3-fluoro-phenyl)-2-(8,8-dimethyl-8-azonia-bicyclo[3.2.1]oct-3-yl)-ethanol iodide;
(3-Endo)-3-[2,2-Bis-(3-fluoro-phenyl)-ethyl]-8,8-dimethyl-8-azonia-bicyclo[3.2.1]octane iodide;
(3-Endo)-3-[2-(3-Chloro-phenyl)-2-phenyl-ethyl]-8,8-dimethyl-8-azonia-bicyclo[3.2.1]octane iodide;
(3-Endo)-3-(2,2-Bis-(3-thienyl)ethyl)-8,8-dimethyl-8-azoniabicyclo[3.2.1]octane iodide;
(3-Endo)-3-[2-Hydroxy-2,2-bis-(3-methyl-2-thienyl)-ethyl]-8,8-dimethyl-8-azonia-bicyclo[3.2.1]octane bromide;
(3-Endo)-3-[2-Hydroxy-2,2-bis-(4-methyl-3-thienyl)-ethyl]-8,8-dimethyl-8-azonia-bicyclo[3.2.1]octane bromide;
(3-Endo)-3-[2-Hydroxy-2,2-bis-(5-methyl-2-thienyl)-ethyl]-8,8-dimethyl-8-azonia-bicyclo[3.2.1]octane bromide;
(3-Endo)-3-{2,2-Bis-[5-(1,1-difluoro-methyl)-2-thienyl]-2-hydroxy-ethyl}-8,8-dimethyl-8-azonia-bicyclo[3.2.1]octane bromide;
(3-Endo)-1,1-Bis-(3-thienyl)-2-(8,8-dimethyl-8-azoniabicyclo[3.2.1]oct-3-yl)ethanol iodide;
(3-Endo)-1,1-bis(3,4-difluorophenyl)-2-(8,8-dimethyl-8-azoniabicyclo[3.2.1]oct-3-yl)ethanol bromide;
(3-Endo)-1,1-bis(3,5-difluorophenyl)-2-(8,8-dimethyl-8-azoniabicyclo[3.2.1]oct-3-yl)ethanol bromide;
(3-Endo)-1,1-dicyclohexyl-2-(8,8-dimethyl-8-azoniabicyclo[3.2.1]oct-3-yl)ethanol bromide;
(3-Endo)-1,1-dicyclopentyl-2-(8,8-dimethyl-8-azoniabicyclo[3.2.1]oct-3-yl)ethanol bromide;
(3-Endo)-1,3-bis(2-fluorophenyl)-2-[(8,8-dimethyl-8-azoniabicyclo[3.2.1]oct-3-yl)methyl]-2-propanol bromide;
2-[(3-(3-Endo))-8,8-dimethyl-8-azoniabicyclo[3.2.1]oct-3-yl]-1,1-di-2-pyridinylethanol iodide;
(3-Endo)-1,1-Bis-(4-fluoro-phenyl)-2-(8,8-dimethyl-8-azonia-bicyclo[3.2.1]oct-3-yl)-ethanol iodide;
(3-Endo)-1,1-Bis-(4-chloro-phenyl)-2-(8,8-dimethyl-8-azonia-bicyclo[3.2.1]oct-3-yl)-ethanol iodide;
(3-Endo)-3-[2,2-Bis-(4-fluoro-phenyl)-ethyl]-8,8-dimethyl-8-azonia-bicyclo[3.2.1]octane iodide;
(3-Endo)-1,1-Bis-(3-chloro-phenyl)-2-(8,8-dimethyl-8-azonia-bicyclo[3.2.1]oct-3-yl)-ethanol iodide;
(3-Endo)-1-(2,3-Fluoro-phenyl)-2-(8,8-dimethyl-8-azonia-bicyclo[3.2.1]oct-3-yl)-1-phenyl-ethanol iodide; and
(3-Endo)-1-(2,3-Chloro-phenyl)-2-(8,8-dimethyl-8-azonia-bicyclo[3.2.1]oct-3-yl)-1-phenyl-ethanol iodide.

4. A pharmaceutical composition comprising a compound according to claim 1 and a pharmaceutically acceptable carrier thereof.

5. (canceled)

6. A method of treating a muscarinic acetylcholine receptor mediated disease, wherein acetylcholine binds to said receptor, comprising administering a safe and effective amount of a compound according to claim 1.

7. A method according to claim 6 wherein the disease is selected from the group consisting of chronic obstructive lung disease, chronic bronchitis, asthma, chronic respiratory obstruction, pulmonary fibrosis, pulmonary emphysema and allergic rhinitis.

8. A method according to claim 7 wherein administration is via inhalation via the mouth or nose.

9. A method according to claim 8 wherein administration is via a medicament dispenser selected from a reservoir dry powder inhaler, a multi-dose dry powder inhaler or a metered dose inhaler.

10. A method according to claim 9 wherein the compound is administered to a human

11. A method according to claim 10 wherein the compound and has a duration of action of 12 hours

12. (canceled)

13. The compound according to claim 1 wherein R1 and R2 are independently selected from group A.

14. The compound according to claim 1 wherein R1 and R2 are independently selected from group B.

15. The compound according to claim 1 wherein R1 and R2 are independently selected from group C.

16. The compound according to claim 1 wherein R1 and R2 are independently selected from benzyl, pyrimidinyl, thiazolyl, isothiazolyl or C3-7 cycloaklyl.

17. The compound according to claim 13 wherein Rb is independently selected from halogen.

18. The compound according to claim 16 wherein s is 1 or 2.

19. The compound according to claim 1 wherein R1 and R2 are selected from 3-hydroxyphenyl, 3-chlorophenyl, 4-chlorophenyl, 3-fluorophenyl, 3,4-difluorophenyl, 3,5-difluorophenyl, 4-fluorophenyl, 2-fluorophenyl, 2,3-difluorophenyl, or 2,3-dichlorophenyl.

20. The compound according to claim 14 wherein Rb is are selected from C1-4 alkyl, halogen, or dihalomethyl.

21. The compound according to claim 19 wherein s is 1 or 2.

22. The compound according to claim 1 wherein R1 and R2 are selected from 5-chloro-thiophen-2-yl, 3-thienyl, 3-methyl-thiophen-2-yl, 4-methyl-thiophen-3-yl, 5-methyl-thiophen-2-yl, or 1,1-difluoromethyl-thiophen-2-yl.

23. The compound according to claim 1 wherein R1 and R2 are selected from pyridine.

24. The compound according to claim 1 wherein R1 and R2 are selected from C3-C7 cycloalkyl.

25. The compound which is:
(3-Endo)-1,1-bis(5-fluoro-2-methylphenyl)-2-(8,8-dimethyl-8-azoniabicyclo[3.2.1]oct-3-yl)ethanol bromide;
(3-Endo)-3-[2-Hydroxy-2,2-bis-(3-methoxy-phenyl)-ethyl]-8,8-dimethyl-8-azonia-bicyclo[3.2.1]octane iodide;
(3-Endo)-3-[2,2-bis(3,4-difluorophenyl)ethyl]-8,8-dimethyl-8-azoniabicyclo[3.2.1]octane bromide;
(3-Endo)-3-[2,2-bis(3,5-difluorophenyl)ethyl]-8,8-dimethyl-8-azoniabicyclo[3.2.1]octane bromide;
(3-Endo)-1,1-bis[5-fluoro-2-(methyloxy)phenyl]-2-(8,8-dimethyl-8-azoniabicyclo[3.2.1]oct-3-yl)ethanol bromide;
(3-Endo)-1,1-bis(3-fluoro-2-methylphenyl)-2-(8,8-dimethyl-8-azoniabicyclo[3.2.1]oct-3-yl)ethanol bromide; or
(3-Endo)-3-[2,2-bis(5-fluoro-2-methylphenyl)ethyl]-8,8-dimethyl-8-azoniabicyclo[3.2.1]octane iodide.

26. A pharmaceutical composition comprising a compound according to claim 25 and a pharmaceutically acceptable carrier thereof.
